(19) United States
(12) Patent Application Publication
(10) Pub. No.: US 2009/0214443 A1
(43) Pub. Date: Aug. 27, 2009

(54) METHOD FOR RETARDING SYSTEMIC DELIVERY RATE FOR EASILY ABSORBABLE ACTIVE AGENTS

(76) Inventor: Thomas Chan, Hopkinton, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/996,812

(22) PCT Filed: Jul. 26, 2006
(86) PCT No.: PCT/US06/28977

§ 371 Date: Apr. 27, 2009

Related U.S. Application Data
(60) Provisional application No. 60/702,998, filed on Jul. 28, 2005.

(51) Int. Cl. A61K 9/12; A61K 31/56

(52) U.S. Cl. 424/45; 514/179

(57) ABSTRACT

Drugs such as hormones, which are easily absorbable through the stratum corneum into the vascular network, are administered to the skin of a patient in an absorption retarding matrix material, thereby retarding the rate at which the drug penetrates into and through the skin.

### BACKGROUND OF THE INVENTION

1. Field of the Invention

The present invention relates to a method for topically delivering certain easily absorbable active agents, including drugs especially lipophilic small molecules, for example, hormones, to a patient in need thereof under conditions which prevent a large spike in the concentration of the hormone in the patient's blood system. More specifically, the invention relates to a method for topically administering a readily absorbable active agent to a patient in need thereof whereby the rate of delivery of the hormone through the stratum corneum and into the vascular network is retarded such that a more desirable, slow and relatively uniform build-up of the active agent in the patient's blood may be achieved.

2. Description of the Related Art

In general, systems designed to deliver testosterone or other androgenic hormones, have been developed with treatment of male patients in mind. It is generally well known, however, that testosterone is relatively easily absorbed into the stratum corneum and epidermis and into and through the dermis where it contacts, penetrates and is taken up into the patient's vascular network to cause a spike or bolus increase in the serum testosterone level. This is especially noticeable when the high ethanol contents of current topical formulations flash off the skin upon application. While the rapid uptake and serum level increase of testosterone may be of physiological benefit to many men in need of testosterone therapy, the same is clearly not the case for women who may require testosterone therapy for their sexual dysfunction.

Accordingly, given the rapid rate at which testosterone in contact with the skin will penetrate into and through the skin and enter the vascular network to increase serum concentrations, it would be beneficial to be able to administer testosterone or other sex hormones or any hormone which is easily absorbed but which requires only a relatively slow, and uniform change in blood serum levels, under conditions by which the rate of administration of the easily absorbable hormone becomes available for systemic delivery but without the attendant side effects associated with elevated levels of the particular hormone or other readily absorbable drug.

### SUMMARY OF THE INVENTION

According to one embodiment, the present invention provides a method for retarding the rate of delivery of an easily-absorbable active agent, comprising, topically administering to said patient a composition comprising an absorption-retarding matrix material and an amount of said active agent; wherein the matrix formed from the matrix-forming material retards the rate at which the active agent penetrates into and through the skin.

According to a further embodiment, the present invention provides a method for retarding the rate of delivery of a lipophilic small molecule, comprising, topically administering to said patient a composition comprising an absorption-retarding matrix material and an amount of said lipophilic small molecule; wherein the matrix formed from the matrix-forming material retards the rate at which the lipophilic small molecule penetrates into and through the skin.

According to yet another embodiment, the present invention provides a method for retarding the rate of delivery of a drug, comprising, topically administering to said patient a composition comprising an absorption-retarding matrix material and an amount of said drug; wherein the matrix formed from the matrix-forming material retards the rate at which the drug penetrates into and through the skin.

According to another embodiment of the invention, a method is provided for retarding the rate of delivery of a normally readily systemically absorbed hormone to a patient in need thereof, comprising, topically administering to said patient a composition comprising a skin-substantive polymerizable matrix material and an amount of said hormone; wherein the matrix formed from the skin-substantive material does not penetrate into or through the skin and substantially preferentially partitions the hormone within the matrix and retards the rate at which the hormone penetrates into and through the skin such that the amount of said hormone which enters into the patient's vascular network is sufficient to achieve its desired physiological or biological function but less than an amount which causes or promotes adverse side effects.

### DETAILED DESCRIPTION OF PREFERRED EMBODIMENTS

Many low molecular weight active agents tend to be readily absorbed through the skin and taken up into the recipient's vascular network. While such rapid absorption and increase in blood concentration may be advantageous for some indications, there are also conditions where rapid increases in or high sustained increases in blood serum level of these agents, may have undesirable side effects for the individual.

One such case is the topical delivery of a hormone, specifically testosterone for treating female sexual dysfunction. Typically, when administered to a male patient to treat hypogonadism or other indications, a large and generally rapid increase in blood serum concentration of testosterone is a main objective to achieve the desired physiological action, e.g., increased libido and mood. However, for application to females, it may be more desirable to avoid a spike in the female's blood testosterone concentration. Rather, a low and slow but steady delivery would be desirable. Accordingly, for many hormones, such as testosterone, commercially existing topical products which are associated with an initial bolus delivery or initial high rate of delivery, would not be safe or useful for application to females to treat female sexual disorders.

Accordingly, the present invention is based on the discovery that certain network or film-forming polymerizable substances that have a higher affinity for the hormone or other easily absorbable drug, such as, for example, small peptides, than does the outer layer of the skin (i.e., the stratum corneum) can provide an effective carrier for such easily absorbable drug molecule for topical application of the easily absorbable drug molecule to the skin. Because the affinity of the drug molecule is higher for the carrier than for the skin, the partition coefficient for determining the distribution of the easily-absorbable molecule between the carrier and the skin favors the former. Accordingly, when a composition comprising the easily-absorbable molecule, e.g., hormone, hormone analogue, small peptides, etc., is deposited on the skin, the tendency for the molecule to be absorbed into and through the skin is counteracted by the stronger affinity for the easily absorbable molecule for the carrier.

It will be appreciated by those skilled in the art that in the embodiments of the present invention by virtue of being incorporated into a matrix having a higher affinity for the hormone or other easily absorbable molecule than between the hormone and the skin, the compositions may be applied directly to the skin without, for example, requiring an intermediary rate-controlling film, as often associated with patch applications for administering drugs. Furthermore, because the matrix material has good adherence when applied to skin (and hair), it is not necessary to use an adhesive layer for achieving the adherence of the topically applied composition to the skin.

In particular, it has been found in accordance with an embodiment of the present invention that one suitable class of absorption-retarding matrix materials is the lipophilic, amphiphilic or hydrophilic film-forming polymers as disclosed in U.S. Pat. No. 5,911,980, the disclosure of which is incorporated herein, in its entirety, by reference thereto; while another suitable class of absorption-retarding matrix materials are the cationic lipophilic, amphiphilic or hydrophilic film-forming polymers, which are disclosed in U.S. Pat. Nos. 5,906,822 and 5,807,957, the disclosures of which are incorporated herein, in their entireties, by reference thereto.

Briefly, these film-forming polymeric materials are characterized by a generally linear, symmetrical structure with terminal hydrophobic hydrocarbon groups or polypropylene groups, linked via polyoxyalkyl groups. The non-ionic compounds as disclosed in U.S. Pat. No. 5,911,980, may be represented by the formula (I):

R(CO)ₘO—(CH₂CHR¹O)ₙ—[OCNH-Z-NHCO₂(CH₂CHR²O)_{n′}]ₚ—OCNH-Z-NHCO₂—(CH₂CHR¹)ₙO(CO)ₘR (I)

where, R represents (i) an alkyl, alkenyl or alkylaryl hydrocarbyl group of from 1 to 30 carbon atoms or (ii) a polypropylene oxide group; R¹ and R², each, independently, represent a hydrogen atom, or a methyl group or ethyl group; Z represents a divalent linking group; m is 0 or 1; n and n′, are each, independently, a positive number; and p is ≧0.

In the above formula (I) the groups R may be bonded through either ether (m=0) or ester (m=1) linkages. In general, ether linkages (m=0) are preferred because of their comparative chemical stability, especially where extremes in pH might be encountered.

The polymers of this invention are film-forming compounds which may be polar/hydrophilic, intermediate polarity/amphiphilic, or non-polar/lipophilic, depending primarily on the choices of R, R¹ and R². In general, larger R, R¹, and R² groups will tend to promote lipophilicity, while the smaller R, R¹, and R² groups will tend to increase hydrophilicity. Combinations of small and large groups among R, R¹ and R² will tend to produce amphiphilicity and consequent surface activity; since R, R¹ and R² can be controlled independently through choice of starting materials, various amphiphilic structures are possible, as will be obvious to those skilled in the art. Exemplary combinations are given in Table 1:

TABLE 1

| R | R¹ | R² | Type |
|---|---|---|---|
| (i) Short hydrocarbyl | H | H | Hydrophilic |
| (ii) Short hydrocarbyl | H | Me, Et | Amphiphilic |
| (iii) Long hydrocarbyl or PPO | H | H | Amphiphilic |
| (iv) Long hydrocarbyl or PPO | Me, Et | Me, Et | Lipophilic |

In Table I “short hydrocarbyl” refers to approximately C₁-C₄ alkyl or alkenyl and “long hydrocarbyl” refers to an approximately C₇-C₃₀ alkyl, alkenyl or alkaryl. For intermediate carbon chain lengths the procedure described below can be used to determine whether any particular polymer is hydrophilic, amphiphilic or lipophilic.

The terms “hydrophilic,” “lipophilic,” and “amphiphilic” refer to relative affinities for, and compatibility with, water versus typical oily/fatty organic materials. A simple test is to physically shake a sample of unknown material with a mixture of both water and a water immiscible organic solvent, such as octanol, until equilibrium is attained, and allow the liquid phases to separate. A substance found preponderantly in the water phase would be judged to be hydrophilic, while conversely a material going to the octanol phase would be considered lipophilic. Amphiphilic substances are those which show a substantial, although not necessarily equal, affinity for both; these will tend to concentrate at the interface of the two phases, characteristically resulting in a reduction in the interfacial tension.

In one embodiment of the invention, the film-forming material is a lipophilic film-forming polymer represented by formula (I-a):

R^{a}(CO)ₘO—(CH₂CHR¹¹O)ₙ—[OCNH-Z-NHCO₂(CH₂CHR¹²O)_{n′}]ₚ—OCNH-Z-NHCO₂—(CH₂CHR¹¹O)ₙ—(CO)ₘR^{a} (I-a)

where R^{a} is an alkyl, alkenyl or alkaryl hydrocarbyl group of from 1 to 30 carbon atoms; Z is a divalent linking group; R¹¹ is a methyl group or ethyl group; R¹² is a methyl group or ethyl group; m is 0 or 1; n is a positive number of at least 2; n′ is a positive number of at least 2; and p is ≧0.

In one embodiment of the invention, the film-forming material is an amphiphilic film-forming polymer represented by the formula (I-b):

R^{c}—(CO)ₘO—{(CH₂CHR¹O)ₙ—)OCNH-Z-NHCO₂(CH₂CHR²O)_{n′}}ₚ—OCNH—NHCO₂—(CH₂CHR¹O)ₙ—(CO)ₘR^{c} (I-b2)

where Z represents a divalent linking group, m is 0 or 1, n and n′, independently, is a positive number≧2, p≧0; R¹ and R², each, independently, represent a hydrogen atom, a methyl group or an ethyl group; and R^{c} represents a short hydrocarbyl group selected from the group consisting of C₁-C₆ alkyl and C₂-C₆ alkenyl, or R^{c} represents a long hydrocarbyl group having from about 7 to about 30 carbon atoms, with the provisos that when R^{c} represents said short hydrocarbyl group one of R¹ or R² (p≠0) is a hydrogen atom and the other is methyl group or ethyl group; and when R^{c} represents said long hydrocarbyl group then R¹ and R² both represent hydrogen atoms.

In one embodiment of the invention, the film-forming material is a hydrophilic film-forming polymer represented by the formula (I-c):

R^{b}(CO)ₘ—O—(CH₂CH₂O)ₙ—[OCNH-Z-NHCO₂(CH₂CH₂O)_{n′}]ₚ—OCNH-Z-NHCO₂—(CH₂CH₂O)ₙ—(CO)ₘR^{b} (I-c)

where Z, m, n, n′ and p have the same meanings as in formula (I-a); and R^{b} represents a short hydrocarbyl group, selected from the group consisting of C₁—C₆ alkyl and C₂-C₆ alkenyl.

The cationic compounds, as disclosed in U.S. Pat. No. 5,807,9957 and U.S. Pat. No. 5,906,822, may be represented by the formula (II):

R(CO)ₘY¹—(CH₂CHR¹O)ₙ—[CONH-Z-NHCO(OCH₂CHR²)_{n′}—Y²—(CH₂CH₂R²O)ₙ]ₚ—CONH-Z-NHCO—(OCH₂CHR¹)ₙY¹(CO)ₘR (II).

In the above formula (II), R represents (i) an alkyl, alkenyl, or alkaryl hydrocarbyl group of from 1 to 30 carbon atoms or (ii) a polypropylene oxide group (PPO) or polybutylene oxide group (PBO); R¹ and R², each, independently, represent a hydrogen atom, or a methyl or ethyl group; Z represents a divalent linking hydrocarbyl group; Y¹ represents O, NR³ or N⁺R³ R⁴X⁻; Y² represents O, NR³, N⁺R³ R⁴X⁻, (R³NCH₂CH₂)ₜ or [(R³R⁴N⁺CH₂CH₂)X]ₜ; R³ and R⁴ independently represent C₁-C₂₂ alkyl and X represents an anion; m is 0 or 1 with the provisos that Y₁ and Y₂ are not both oxygen (O) at the same time and that when Y¹=NR³ or N⁺R³R⁴X⁻, then m=0; n, n′ and n″, are each, independently, a positive number; p is ≧0, preferably p is 0 or 1, and t is a positive integer. Most preferably, n, n′, n″ and p are selected such that 2 n+pn′+pn″ is at least about 20.

The groups R in formula (II) may be bonded to the polyalkyleneoxy group (CH₂CHR¹O) through either ether (m=0) or ester (m=1) linkages, when y¹ is O. In general, ether linkages (m=0) are preferred because of their comparative chemical stability, especially where extremes in pH might be encountered. When Y^{.sup.}1 represents the amine group NR³ or quaternary ammonium group N⁺R³ R⁴X⁻, then m=0. Preferably, Y¹ represents NR³ or N⁺R³R⁴X⁻).

The amphiphilic, hydrophilic and lipophilic compounds represented by formula (II) are film-forming cationic polymeric type compounds and have a generally linear, symmetrical structure with terminal hydrophobic hydrocarbon groups or polyalkylene oxide groups which, in one embodiment, are linked to the central urethanyl core groups via polyoxyalkyl amine groups or quaternized polyoxyalkyl amine groups; and, in an alternative embodiment, polyoxyalkylamine groups or quaternized polyoxyalkylamine groups link together plural urethanyl groups. It is also possible to provide the polymers with the tertiary amine or quaternary amine groups linking together the terminal groups to the central urethanyl core groups as well as linking together plural urethanyl groups.

As used herein, the term “cationic” is intended to include the compounds with the polyoxyalkyl amine linking groups and which are cationic under acidic pH conditions as well as the quaternary group containing compounds.

The factors discussed above for the polymers of formula (I) with regard to the “philicity” characteristics apply equally for the polymers of formula (II).

Compounds of formula (I) and formula (II) are available, under the trademark, MacroDerm®, from MacroChem Corporation, Lexington, Mass.

In view of their relatively high molecular weights, (generally from about 1,000 to about 30,000) and the repeating polyoxyalkyl and diurethane units, the compounds of formula (I) and (II) may be referred to polymeric or polymer products, with the understanding, however, that after application to the patient, the compounds may undergo further association into larger polymers or as substantially occlusive networks of such compounds. Especially within the more preferred molecular weight range of from about 1,000 to 25,000, more preferably, from about 2,000 to about 22,000, even more preferably, from about 2,000 to about 15,000 (Le., wherein 2n+pn′ ranges from about 20 to about 150), the polymers are substantive to skin, and provide occlusion and/or barrier properties, while themselves showing little or no actual penetration into the skin. Depending on the selection of, primarily, the polyoxyalkyl groups, (i.e., on R¹ and R²), the polymers may exhibit one or more of the following properties: humectancy, wetting, emolliency, solubilization.

Similarly, the selection of the terminal hydrocarbyl group R will affect the properties of the polymer. For instance in the hydrophilic type (i) the preferred C₁-C₄ alkyl or C₂-C₄ alkenyl groups are only large enough to cap the hydroxyl groups provided by the polyoxyethylene group (R¹=R²=H). Such compounds are considered truly hydrophilic. Conversely, longer alkyl, alkenyl, alicyclic, or alkaryl (R) terminal groups ensure that the polymer will, depending on R¹ and R², be amphiphilic and show surface activity (R¹ or R²═H, type (iii) in Table 1), or be essentially lipophilic (R¹ and R²═Me or Et, type (iv) in Table 1).

The urethane linkages used for coupling the polyoxyalkyl groups are stable and biocompatible and may also contribute to physical properties. For example, in solution in appropriate solvents or carriers, the urethane groups may exhibit intermolecular hydrogen bonding and, when applied topically, may provide specific bonding with skin proteins.

The bridging group Z (preferably alkylene or cycloalkylene of from 2 to about 16 carbon atoms) provides one or more sites of either relative rigidity or flexibility.

In general, the component groups of the compounds of formula (I) and (II) are derived from starting materials which are themselves widely accepted for use in the cosmetic, pharmaceutical and biomedical polymer areas.

The compositions for topical application will include the hormone or analogue thereof, or a small peptide or other drug which is readily or easily absorbable into and through the skin of the individual in need of treatment. In this regard, the term “readily or easily absorbable,” or similar terminology, refers to such drugs which when topically applied to the skin in the form of a solution thereof, will be absorbed into and through the stratum corneum, epidermis and dermis, without pre-treatment of the skin and without the aid or assistance of a skin-penetration enhancing agent.

The absorption-retarding matrix material, together with the readily or easily absorbable hormone or similar active agent, may be dispersed within a suitable topical carrier. The topically applied compositions are effective for enhancing adherence of the hormone on and to the skin and effectively retard or modulate the rate at which it will be absorbed into and through the skin.

By “topical administration” or “dermal administration” is meant the local, administration of the easily or readily absorbable hormone on the skin, that is, without causing immediate or rapid passage of the hormone into the blood stream and maintaining the blood serum level of the hormone at a safe and effective level for minimizing unwanted (adverse) side effects yet sufficient to exhibit the desired physiological effect. For example, in the case of topical administration of testosterone to females, the method of the embodiments of the present invention can be used to assure that only an amount of testosterone which is effective to, for example, treat the symptoms of female sexual dysfunction are reached within up to about 2 to 4 hours, for example, but can remain in place at the site of administration to continually administer the testosterone at acceptable levels for one or more days without causing the blood serum levels of testosterone to cause local or systemic unwanted side effects, such as, for example, unwanted hair growth.

Thus, in general, by “effective” amount of the easily-absorbable hormone or other easily absorbable active agent, is meant a nontoxic but sufficient amount of the active agent, to provide the desired local/systemic effect and performance at a reasonable benefit/risk ratio attending any medical treatment, while substantially eliminating or reducing the risk of unwanted side effects associated with higher local and blood concentrations of the active agent.

“Topical carriers” as used herein refer to vehicles suitable for topical applications of drugs or cosmetics, and include any such liquid or non-liquid solvent, diluent or the like materials known in the cosmetic and medical arts, for forming any liquid or semisolid gel, cream, ointment, emulsion, aerosol, foam, lotion, or the like, and which does not adversely affect living animal tissue or interact with other components of the composition in a deleterious manner. Topical carriers are used to provide the compositions of the invention in their preferred liquid, topically administrable form. Examples of suitable topical carriers for use herein include water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials, and mixtures thereof.

The following examples illustrate embodiments showing the retarded delivery of testosterone from a representative matrix forming polymerizable carrier, but it will be appreciated that these are presented only for assisting the understanding of the present invention, and are not to be construed as limiting the scope thereof.

### EXAMPLES

The following formulations were prepared as alcoholic (ethanol) solutions of 1 wt % testosterone, with or without a polymerizable matrix-forming material, MacroDerm® (0, 5, 10 or 15 wt %), according to an embodiment of the invention.

Each formulation was applied to an area of approximately 0.7 sq·cm of excised human skin mounted on a Franz diffusion cell and the concentration of testosterone remaining in the matrix and the concentration of testosterone absorbed into and through the skin and available to come into contact with the vasculature for uptake therein, were also measured, to determine cumulative delivery (% dose) and flux (μg/cm²/hr). The results are shown in Tables 2 and 3 below.

The MacroDerm film-forming polymer used in these examples were substantially the same as the amphiphilic oligomers of Example 1 in U.S. Pat. No. 5,911,980, wherein R=stearyl; twenty (20) ethylene oxide main chains; —OCHN-Z-NHCO₂ represents methylenebiscyclohexyl diisocyanate coupling unit; and two (2) ethylene oxide central chains.

TABLE 2
Flux, average

| | | 1% | | |
|---|---|---|---|---|
| | 1% | Testosterone | 1% | |
| Time | Testosterone | 5% | Testosterone | 1% Testosterone |
| (h) | 99% EtOH | MacroDerm | 10% MacroDerm | 15% MacroDerm |
| 0 | 0.00 | 0.00 | 0.00 | 0.00 |
| 1 | 1.00 | 0.40 | 0.39 | 0.37 |
| 3 | 1.43 | 0.70 | 0.65 | 0.55 |
| 5 | 1.29 | 0.74 | 0.66 | 0.55 |
| 7 | 1.32 | 0.77 | 0.72 | 0.70 |
| 16 | 0.76 | 0.59 | 0.56 | 0.51 |

TABLE 3
Cumulative Amount (%), average

| | | 1% | | |
|---|---|---|---|---|
| | 1% | Testosterone | 1% | 1% |
| Time | Testosterone, | 5% | Testosterone | Testosterone |
| (h) | 99% EtOH | MacroDerm | 10% MacroDerm | 15% MacroDerm |
| 0 | 0.00 | 0.00 | 0.00 | 0.00 |
| 2 | 2.32 | 0.92 | 0.90 | 0.83 |
| 4 | 5.60 | 2.51 | 2.39 | 2.11 |
| 6 | 8.56 | 4.21 | 3.90 | 3.37 |
| 8 | 11.59 | 5.97 | 5.54 | 4.98 |
| 24 | 25.54 | 16.68 | 15.85 | 14.38 |

In Table 3, the cumulative dose is expressed as % of applied dose (to the skin) that has accumulated in the receptor phase of a Franz cell. These solutions cover the skin area to which they are applied and were applied at a dose of ˜7 μL/0.7 cm² to allow formation of a non-occlusive network or film of the MacroDerm polymer carrier covering the skin (human cadaver skin from back and/or abdomen, were used) after the ethanol has evaporated at 32° C. skin temperature.

What is claimed is:

1. A method for retarding the rate of delivery of an easily-absorbable hormone, comprising,
topically administering to said patient a composition comprising an absorption-retarding matrix material and an amount of said hormone;
wherein the matrix formed from the matrix-forming material retards the rate at which the hormone penetrates into and through the skin.

2. A method according to claim 1 wherein the composition further comprises a vehicle suitable for topical drug delivery.

3. A method according to claim 1, wherein the easily-absorbable hormone is testosterone; wherein the patient is a female mammal.

4. A method according to claim 1, wherein the absorption-retarding matrix material is a compound having the formula (I):
R(CO)ₘO—(CH₂CHR¹O)ₙ—[OCNH-Z-NHCO₂(CH₂CHR²O)_{n′}]ₚ—OCNH-Z-NHCO₂—(CH₂CHR¹)ₙO(CO)ₘR (I)
wherein, R represents (i) an alkyl, alkenyl or alkylaryl hydrocarbyl group of from 1 to 30 carbon atoms or (ii) a polypropylene oxide group; R¹ and R², each, independently, represent a hydrogen atom, or a methyl group or ethyl group; Z represents a divalent linking group; m is 0 or 1; n and n′, are each, independently, a positive number; and p is ≧0.

5. A method according to claim 1, wherein the absorption-retarding matrix material is an amphiphilic compound having the formula (I-b):
R^{c}—(CO)ₘO—{(CH₂CHR¹O)ₙ—)OCNH-Z-NHCO₂(CH₂CHR²O)_{n′}}ₚ—OCNH—NHCO₂—(CH₂CHR¹O)ₙ—(CO)ₘR^{c} (I-b)
where Z represents a divalent linking group, m is 0 or 1, n and n′, independently, is a positive number≧2, p≧0; R¹ and R², each, independently, represent a hydrogen atom, a methyl group or an ethyl group; and R^{c} represents a short hydrocarbyl group selected from the group consisting of C₁-C₆ alkyl and C₂-C₆ alkenyl, or R^{c} represents a long hydrocarbyl group having from about 7 to about 30 carbon atoms, with the provisos that when R^{c} represents said short hydrocarbyl group one of R¹ or R² (p≠0) is a hydrogen atom and the other is methyl group or ethyl group; and when R^{c} represents said long hydrocarbyl group then R¹ and R² both represent hydrogen atoms.

6. A method according to claim 1, wherein the composition comprises a liquid or non-liquid topical carrier and is in the form of a solution, lotion, gel, cream, ointment, aerosol, spray or emulsion.

7. A method for retarding the rate of delivery of a normally readily systemically absorbed hormone to a patient in need thereof, comprising,
topically administering to said patient a composition comprising a skin-substantive polymerizable matrix material and an amount of said hormone;
wherein the matrix formed from the skin-substantive material does not penetrate into or through the skin and substantially preferentially partitions the hormone in the body thereof and retards the rate at which the hormone penetrates into and through the skin such that the amount of said hormone which enters into the patient's vascular network is sufficient to achieve its desired physiological or biological function but less than an amount which causes or promotes adverse side effects.

8. A method according to claim 7, for treating female sexual dysfunction, which comprises topically administering to a female individual in need thereof, a composition comprising a physiologically effective amount of testosterone and an amphiphilic matrix-forming material having the formula (I-b):
R^{c}—(CO)ₘO—{(CH₂CHR¹O)ₙ—)OCNH-Z-NHCO₂(CH₂CHR²O)_{n′}}ₚ—OCNH—NHCO₂—(CH₂CHR¹O)ₙ—(CO)ₘR^{c} (I-b)
where Z represents a divalent linking group, m is 0 or 1, n and n′, independently, is a positive number≧2, p≧0; R¹ and R², each, independently, represent a hydrogen atom, a methyl group or an ethyl group; and R^{c} represents a short hydrocarbyl group selected from the group consisting of C₁-C₆ alkyl and C₂-C₆ alkenyl, or R^{c} represents a long hydrocarbyl group having from about 7 to about 30 carbon atoms, with the provisos that when R^{c} represents said short hydrocarbyl group one of R¹ or R² (p≠0) is a hydrogen atom and the other is methyl group or ethyl group; and when R^{c} represents said long hydrocarbyl group then R¹ and R² both represent hydrogen atoms; and a vehicle suitable for topical drug delivery, whereby such composition adheres to the skin of the female individual for a period of time sufficient for the individual's blood serum testosterone level to increase to a desired amount but without reaching a level associated with adverse side effects.
